(19) United States
(12) Patent Application Publication
(10) Pub. No.: US 2026/0240423 A1
(43) Pub. Date: Aug. 20, 2026

(54) INSTRUMENT CHANNEL TUBE, INSERTION SECTION, AND ENDOSCOPE

(71) Applicant: HUNAN VATHIN MEDICAL INSTRUMENT CO., LTD., Xiangtan (CN)

(72) Inventors: Xihuan Yin, Xiangtan (CN); Qinghua Yi, Xiangtan (CN)

(73) Assignee: HUNAN VATHIN MEDICAL INSTRUMENT CO., LTD., Xiangtan (CN)

(21) Appl. No.: 19/641,556

(22) Filed: Apr. 8, 2026

Related U.S. Application Data
(63) Continuation of application No. PCT/CN2024/130197, filed on Nov. 6, 2024.

(30) Foreign Application Priority Data
Oct. 9, 2023 (CN) 202311294526.0

(51) Int. Cl. A61B 1/018; A61B 1/00

(52) U.S. Cl. CPC A61B 1/018; A61B 1/00128

(57) ABSTRACT

An instrument channel tube, an insertion section, and an endoscope, relating to the field of medical devices. The instrument channel tube includes an instrument channel tube body having an instrument channel formed therein. A proximal end of the instrument channel tube body is sleeve-fitted with a connector head at a distal end of a three-way connector of an endoscope, such that the instrument channel is in communication with a suction port and an instrument port on the three-way connector. A first adhesive layer is disposed between the connector head and the instrument channel tube body. The instrument channel tube further includes a coil spring tube disposed at a sleeve-fit junction between the instrument channel tube body and the connector head, the coil spring tube being configured to apply a preload to the sleeve-fit junction between the instrument channel tube body and the connector head.

### CROSS-REFERENCE TO RELATED APPLICATION

This application is a U.S. National Stage application of International Application No. PCT/CN2024/130197, filed on November 6, 2024, which claims the benefit of priority to Chinese Patent Application No. CN202311294526.0, filed on October 9, 2023. The right of priority to Chinese Patent Application No. CN202311294526.0 was restored by the Receiving Office (RO/CN) pursuant to PCT Rule 26bis.3. The entire disclosures of the aforementioned applications are incorporated herein by reference.

### TECHNICAL FIELD

The present disclosure relates to the field of medical devices, and more particularly, to an instrument channel tube, an insertion section, and an endoscope.

### BACKGROUND

An endoscope is a commonly used medical device for examination, diagnosis, and treatment of diseases. An endoscope typically includes an insertion section configured for insertion into the interior of a human body, and a handle connected to the insertion section.

An instrument channel tube is disposed within the insertion section of the endoscope. A hollow interior of the instrument channel tube defines an instrument channel configured to allow passage of treatment instruments therethrough. In the related art, a proximal end of the instrument channel tube is connected to the handle by means of adhesive bonding. Adhesive bonding offers the advantages of low cost and good sealing performance, and is applicable to structures of various shapes, providing high versatility. However, the inventor has found that an instrument channel tube made of polytetrafluoroethylene (PTFE) material has poor adhesion properties, resulting in low reliability of the connection between the instrument channel tube and the handle.

Therefore, providing an endoscope in which the connection between the instrument channel tube and the handle is reliable constitutes a technical problem to be solved by those skilled in the art.

### SUMMARY

The present disclosure provides an instrument channel tube, an insertion section, and an endoscope, to solve the technical problem in the related art that poor adhesion properties of the instrument channel tube lead to low reliability of the connection between the instrument channel tube and the handle.

To solve the above problem, the present disclosure adopts the following technical solutions:

A first aspect of the present disclosure provides an instrument channel tube.

The instrument channel tube of the present disclosure is for use in an endoscope. The instrument channel tube includes an instrument channel tube body having an instrument channel formed therein. A proximal end of the instrument channel tube body is sleeve-fitted with a connector head at a distal end of a three-way connector of the endoscope, such that the instrument channel is in communication with a suction port and an instrument port on the three-way connector. A first adhesive layer is further disposed between the connector head and the instrument channel tube body. The instrument channel tube further includes a coil spring tube disposed at a sleeve-fit junction between the instrument channel tube body and the connector head, the coil spring tube maintaining an interference fit with the sleeve-fit junction between the instrument channel tube body and the connector head, thereby applying a preload to the sleeve-fit junction between the instrument channel tube body and the connector head via the coil spring tube.

In a further embodiment, the proximal end of the instrument channel tube body is sleeve-fitted over an exterior of the connector head, and the coil spring tube is sleeve-fitted over an exterior of the instrument channel tube body.

In a further embodiment, at least one of the connector head and the proximal end of the instrument channel tube body has a tapered configuration. A diameter of the tapered configuration gradually decreases in a direction from the proximal end toward the distal end. An inner diameter of a proximal end of the coil spring tube is less than an outer diameter of the tapered configuration, such that when the coil spring tube is sleeve-fitted over the sleeve-fit junction between the instrument channel tube body and the connector head, the coil spring tube at the tapered configuration is configured to be radially expanded.

In a further embodiment, the coil spring tube extends from the proximal end of the instrument channel tube body toward the distal end of the instrument channel tube body, and the coil spring tube extends at least to a bending section of the instrument channel tube body.

In a further embodiment, a pitch of the coil spring tube is less than or equal to a diameter of the instrument channel tube body.

In a further embodiment, a first strength of the coil spring tube at the bending section is greater than a second strength of the coil spring tube at a non-bending section; and/or a first pitch of the coil spring tube at the bending section is less than a second pitch of the coil spring tube at the non-bending section.

In a further embodiment, a second adhesive layer is further disposed between the coil spring tube and the connector head, or between the coil spring tube and the instrument channel tube body.

In a further embodiment, the instrument channel tube further includes an overmold layer disposed over and encapsulating an exterior of the proximal end of the coil spring tube. The overmold layer extends from the proximal end of the coil spring tube toward the distal end of the coil spring tube, and extends at least to a distal end portion of the coil spring tube.

A second aspect of the present disclosure provides an insertion section.

The insertion section of the present disclosure is for use in an endoscope. The insertion section includes an insertion section body and an instrument channel tube, wherein the instrument channel tube is the instrument channel tube according to any one of the technical solutions of the present disclosure. An accommodation space is defined within the insertion section body and extends along an axial direction of the insertion section body. The instrument channel tube is disposed within the accommodation space.

A third aspect of the present disclosure provides an endoscope.

The endoscope of the present disclosure includes an insertion section and a handle, wherein the insertion section is the insertion section according to any one of the technical solutions of the present disclosure. A three-way connector is disposed within the handle. The three-way connector has a first port, a second port, and a third port. The first port is in communication with the connector head, the second port is in communication with the suction port, and the third port is in communication with the instrument port.

The technical solutions adopted by the present disclosure achieve the following beneficial effects:

In the instrument channel tube of the present disclosure, which is for use in an endoscope, the proximal end of the instrument channel tube body is sleeve-fitted with the connector head at the distal end of the three-way connector of the endoscope, and a first adhesive layer is disposed between the instrument channel tube body and the connector head. Thus, in the instrument channel tube of the present disclosure, the instrument channel tube body can be fixedly connected to the connector head via the first adhesive layer. Specifically, adhesive can be dispensed between the instrument channel tube body and the connector head by means of adhesive dispensing, with the adhesive forming the first adhesive layer to fixedly connect the instrument channel tube body and the connector head. This approach not only provides the advantages of simple operation and being unaffected by the shapes and dimensions of the instrument channel tube body and the connector head, but also allows the first adhesive layer to extend circumferentially around the entire junction between the instrument channel tube body and the connector head, providing good sealing performance.

The instrument channel tube of the present disclosure further includes the coil spring tube. The coil spring tube is disposed at the sleeve-fit junction between the instrument channel tube body and the connector head. The preload applied to the sleeve-fit junction between the instrument channel tube body and the connector head via the coil spring tube enhances the stability of the adhesive bond between the instrument channel tube body and the connector head, thereby improving the reliability of the connection at the junction between the instrument channel tube body and the connector head. This solves the technical problem in the related art that poor adhesion properties of the instrument channel tube lead to low reliability of the connection between the instrument channel tube and the handle. Additionally, when the insertion section of the endoscope is bent, the junction between the instrument channel tube body and the connector head also undergoes passive bending. Because the coil spring tube is flexible, when bending occurs at the junction between the instrument channel tube body and the connector head, the coil spring tube bends accordingly while still applying a preload to the junction between the instrument channel tube body and the connector head, such that the reliability of the connection at the junction between the instrument channel tube body and the connector head is unaffected by bending of the insertion section.

### BRIEF DESCRIPTION OF THE DRAWINGS

To more clearly illustrate the technical solutions of the embodiments of the present disclosure or the prior art, the following briefly introduces the drawings used in the description of the embodiments or the prior art. It is apparent that the drawings described below are merely some embodiments of the present disclosure, and that other drawings may be derived from these drawings by those of ordinary skill in the art without creative effort.

FIG. 1 is a first schematic structural view illustrating the insertion section of the endoscope according to an embodiment of the present application in a bent state;

FIG. 2 is a second schematic structural view illustrating the insertion section of the endoscope according to the embodiment of the present application in a bent state;

FIG. 3 is a third schematic structural view illustrating the insertion section of the endoscope according to the embodiment of the present application in a bent state;

FIG. 4 is a schematic structural view illustrating the insertion section of the endoscope according to the embodiment of the present application in a straightened state;

FIG. 5 is an enlarged view of portion A in FIG. 4;

FIG. 6 is a first schematic structural view of the three-way connector of the endoscope according to the embodiment of the present application;

FIG. 7 is a second schematic structural view of the three-way connector of the endoscope according to the embodiment of the present application;

FIG. 8 is a cross-sectional view taken along line B-B of FIG. 7.

### REFERENCE NUMERALS

100, instrument channel tube; 101, instrument channel tube body; 1011, tapered configuration; 1012, bending section; 1012a, compression side; 1012b, tension side; 1013, non-bending section; 102, coil spring tube; 103, overmold layer; 200, three-way connector; 201, connector head; 2011, first port; 202, suction port; 2021, second port; 203, instrument port; 2031, third port.

### DETAILED DESCRIPTION

To make the objects, technical solutions, and advantages of the present disclosure clearer, the technical solutions of the present disclosure will be described in detail below. It is apparent that the described embodiments are merely some, but not all, embodiments of the present disclosure. All other embodiments obtained by those of ordinary skill in the art based on the embodiments of the present disclosure without creative effort fall within the scope of protection of the present disclosure.

The terms "first," "second," and the like in the specification and claims of the present application are used to distinguish similar objects and are not used to describe a specific order or sequence. It should be understood that the terms used in this manner are interchangeable where appropriate, so that the embodiments of the present application can be implemented in an order other than that illustrated or described herein, and the objects distinguished by "first," "second," and the like are generally of the same category and do not limit the number of objects. For example, a first object may be one or more. In addition, "and/or" in the specification and claims indicates at least one of the connected objects, and the character "/" generally indicates an "or" relationship between the associated objects.

In the embodiments of the present application, the terms "proximal end" and "distal end" refer to positions of respective components relative to a user during use. An end closer to the user is designated as the "proximal end," and an end farther from the user is designated as the "distal end."

In the related art, an endoscope includes an insertion section and a handle. A three-way connector is disposed within a housing of the handle and is configured to connect a connector head, a suction port, and an instrument port. An instrument channel tube is disposed within the insertion section. A hollow interior of the instrument channel tube defines an instrument channel configured to allow passage of treatment instruments therethrough. A proximal end of the instrument channel tube is connected to the connector head at a distal end of the three-way connector by means of adhesive bonding. The instrument channel tube is typically made of polytetrafluoroethylene (PTFE) material, which has poor adhesion properties, resulting in low reliability of the connection between the instrument channel tube and the three-way connector. Meanwhile, during bending of the insertion section of the endoscope, the junction between the instrument channel tube and the three-way connector may also undergo bending, posing a risk of disconnection of the instrument channel tube from the three-way connector at the junction. In particular, for endoscopes having a relatively short insertion section (such as a nephroscope, whose insertion section has a length of only approximately 30 cm), the risk of disconnection at the junction between the instrument channel tube and the three-way connector is especially pronounced during bending of the insertion section.

Accordingly, the present application provides an instrument channel tube, an insertion section, and an endoscope. The instrument channel tube includes an instrument channel tube body. The instrument channel tube body is fixedly connected to the connector head at the distal end of the three-way connector via an adhesive layer. The instrument channel tube further includes a coil spring tube. The coil spring tube is disposed at the sleeve-fit junction between the instrument channel tube body and the connector head. The coil spring tube clamps the sleeve-fit junction between the instrument channel tube body and the connector head, i.e., applies a preload to the sleeve-fit junction between the instrument channel tube body and the connector head, thereby not only improving the reliability of the connection at the junction between the instrument channel tube body and the connector head, but also ensuring that the reliability of the connection at the junction between the instrument channel tube body and the connector head is unaffected by bending of the insertion section.

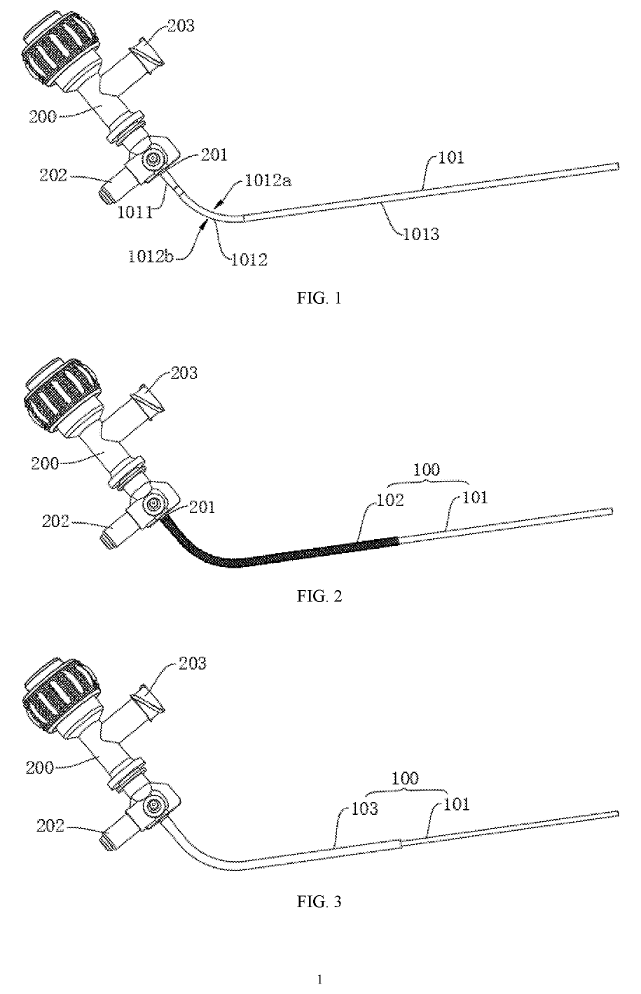

The instrument channel tube, insertion section, and endoscope provided by the embodiments of the present application are described in detail below with reference to FIGS. 1 through 8 by way of specific embodiments and application scenarios thereof.

In one aspect, the present embodiment provides an instrument channel tube for use in an endoscope.

The instrument channel tube of the present embodiment includes an instrument channel tube body 101, as illustrated in FIGS. 1 through 4. The instrument channel tube body 101 has a hollow interior that defines an instrument channel. The instrument channel is configured to receive treatment instruments inserted therethrough, or to inject fluid into the body or aspirate foreign matter from the body.

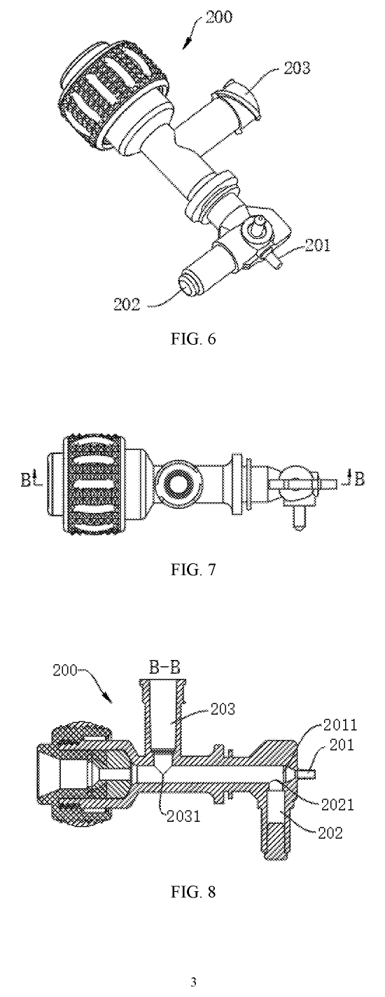

A three-way connector 200 is disposed within the handle housing of the endoscope, and three ports of the three-way connector 200 are respectively connected to a connector head 201, a suction port 202, and an instrument port 203, as illustrated in FIGS. 6 through 8.

The proximal end of the instrument channel tube body 101 is sleeve-fitted with the connector head 201 at the distal end of the three-way connector 200 of the endoscope, such that the instrument channel is in communication with the suction port 202 and the instrument port 203 on the three-way connector 200. A first adhesive layer is further disposed between the connector head 201 and the instrument channel tube body 101, as illustrated in FIGS. 1 through 4 and FIGS. 6 through 8. Preferably, the first adhesive layer may be a bonding layer formed by adhesive. The first adhesive layer is not shown in the drawings. Specifically, after the proximal end of the instrument channel tube body 101 is sleeve-fitted with the connector head 201, adhesive can be dispensed between the instrument channel tube body 101 and the connector head 201 by means of adhesive dispensing, with the adhesive forming the first adhesive layer to fixedly connect the instrument channel tube body 101 and the connector head 201.

After the instrument channel tube body 101 is sleeve-fitted with the connector head 201, the instrument channel is in communication with the suction port 202 and the instrument port 203 on the three-way connector 200, such that foreign matter within the body can be aspirated through the suction port 202, and treatment instruments can be inserted or fluid can be injected into the body through the instrument port 203.

The instrument channel tube of the present embodiment further includes a coil spring tube 102, as illustrated in FIG. 2. The coil spring tube 102 is disposed at the sleeve-fit junction between the instrument channel tube body 101 and the connector head 201, and the coil spring tube 102 maintains an interference fit with the sleeve-fit junction between the instrument channel tube body 101 and the connector head 201, thereby applying a preload to the sleeve-fit junction between the instrument channel tube body 101 and the connector head 201 via the coil spring tube 102, as illustrated in FIG. 2. Specifically, an inner diameter of the coil spring tube 102 is less than the dimension of the sleeve-fit junction between the instrument channel tube body 101 and the connector head 201, enabling the coil spring tube 102 to maintain an interference fit with the sleeve-fit junction between the instrument channel tube body 101 and the connector head 201, thereby providing a preload to the sleeve-fit junction between the instrument channel tube body 101 and the connector head 201. The preload in the present embodiment may also be understood as a compressive force, i.e., the coil spring tube 102 clamps the sleeve-fit junction between the instrument channel tube body 101 and the connector head 201.

Preferably, the instrument channel tube body 101 may be sleeve-fitted over the exterior of the connector head 201, in which case the coil spring tube 102 is sleeve-fitted over the exterior of the instrument channel tube body 101, as illustrated in FIGS. 2 through 5; alternatively, the connector head 201 may be sleeve-fitted over the exterior of the instrument channel tube body 101, in which case the coil spring tube 102 is sleeve-fitted over the exterior of the connector head 201.

In the above embodiment, the proximal end of the instrument channel tube body 101 is sleeve-fitted with the connector head 201 at the distal end of the three-way connector 200 of the endoscope, and the first adhesive layer is disposed between the instrument channel tube body 101 and the connector head 201. Thus, in the instrument channel tube of the present embodiment, the instrument channel tube body 101 can be fixedly connected to the connector head 201 via the first adhesive layer. Specifically, adhesive can be dispensed between the instrument channel tube body 101 and the connector head 201 by means of adhesive dispensing, with the adhesive forming the first adhesive layer to fixedly connect the instrument channel tube body 101 and the connector head 201. This approach not only provides the advantages of simple operation and being unaffected by the shapes and dimensions of the instrument channel tube body 101 and the connector head 201, but also allows the first adhesive layer to extend along the entire circumferential direction of the instrument channel tube body 101 and the connector head 201, providing good sealing performance.

The instrument channel tube of the present embodiment further includes the coil spring tube 102. The coil spring tube 102 is disposed at the sleeve-fit junction between the instrument channel tube body 101 and the connector head 201. The preload applied to the sleeve-fit junction between the instrument channel tube body 101 and the connector head 201 via the coil spring tube 102 enhances the stability of the adhesive bond between the instrument channel tube body 101 and the connector head 201, thereby improving the reliability of the connection at the junction between the instrument channel tube body 101 and the connector head 201. This solves the technical problem in the related art that poor adhesion properties of the instrument channel tube lead to low reliability of the connection between the instrument channel tube and the three-way connector 200. Additionally, when the insertion section of the endoscope is bent, the junction between the instrument channel tube body 101 and the connector head 201 also undergoes passive bending. Because the coil spring tube 102 is flexible, when bending occurs at the junction between the instrument channel tube body 101 and the connector head 201, the coil spring tube 102 bends accordingly while still applying a preload to the junction between the instrument channel tube body 101 and the connector head 201, such that the reliability of the connection at the junction between the instrument channel tube body 101 and the connector head 201 is unaffected by bending of the insertion section.

According to a preferred embodiment, the proximal end of the instrument channel tube body 101 is sleeve-fitted over the exterior of the connector head 201, and the coil spring tube 102 is sleeve-fitted over the exterior of the instrument channel tube body 101. Preferably, the connector head 201 may be made of a material having a greater hardness than that of the instrument channel tube body 101. In the instrument channel tube of the present preferred embodiment, on one hand, the coil spring tube 102 tightly cinches the instrument channel tube body 101 against the exterior of the connector head 201, enhancing the reliability of the adhesive bond between the instrument channel tube body 101 and the connector head 201; on the other hand, the connector head 201 provides support for the instrument channel tube body 101, preventing deformation of the proximal end of the instrument channel tube body 101 due to the compressive force exerted by the coil spring tube 102, which would otherwise affect the patency of the instrument channel.

According to a preferred embodiment, at least one of the connector head 201 and the proximal end of the instrument channel tube body 101 has a tapered configuration 1011, and a diameter of the tapered configuration 1011 gradually decreases in a direction from the proximal end toward the distal end. Parameters such as the taper ratio and diameter of the tapered configuration 1011 may be determined based on the length of the insertion section of the endoscope, provided that a diameter at the distal end of the tapered configuration 1011 is the same as a dimension of the proximal end of the instrument channel tube body 101. When the connector head 201 has the tapered configuration 1011, the proximal end of the instrument channel tube body 101 may be a cylindrical structure having a uniform diameter throughout. When the instrument channel tube body 101 is sleeve-fitted over the exterior of the connector head 201, the proximal end of the instrument channel tube body 101 is expanded and closely conforms to the exterior of the connector head 201. When the connector head 201 has the tapered configuration 1011, the proximal end of the instrument channel tube body 101 may alternatively have a configuration matching the tapered configuration 1011, such that the proximal end of the instrument channel tube body 101 is configured to mate with the connector head 201. Similarly, when the proximal end of the instrument channel tube body 101 has the tapered configuration 1011, the connector head 201 may also be a cylindrical structure having a uniform diameter throughout, or a structure matching the tapered configuration 1011. FIGS. 1 and 5 illustrate an example in which the proximal end of the instrument channel tube body 101 has the tapered configuration 1011.

The inner diameter of the proximal end of the coil spring tube 102 is less than the outer diameter of the tapered configuration 1011, such that when the coil spring tube 102 is sleeve-fitted over the sleeve-fit junction between the instrument channel tube body 101 and the connector head 201, the coil spring tube 102 at the tapered configuration 1011 is radially expanded. In the instrument channel tube of the present preferred embodiment, when the coil spring tube 102 is engaged in position, the coil spring tube 102 is radially expanded at the tapered configuration 1011, such that the coil spring tube 102 exerts a reactive force on the instrument channel tube body 101 or the connector head 201, clamping the instrument channel tube body 101 and the connector head 201 together, thereby further improving the reliability of the connection at the junction between the instrument channel tube body 101 and the connector head 201.

According to a preferred embodiment, the coil spring tube 102 extends from the proximal end of the instrument channel tube body 101 toward the distal end of the instrument channel tube body 101, and the coil spring tube 102 extends at least to the bending section 1012 of the instrument channel tube body 101, as illustrated in FIG. 2. Preferably, a first length of the coil spring tube 102 is greater than a second length from the proximal end of the instrument channel tube body 101 to the bending section 1012, as illustrated in FIG. 2. The bending section 1012 refers to a section of the instrument channel tube body 101 where bending occurs, as illustrated in FIG. 1. As illustrated in FIG. 1, the compression side 1012a is the compressed side of the bending section 1012, and the tension side 1012b is the stretched side.

In the instrument channel tube of the present preferred embodiment, the coil spring tube 102 has superior deformation characteristics compared to the instrument channel tube body 101, maintaining its circular circumferential shape even during bending. When the instrument channel tube body 101 undergoes bending, the coil spring tube 102 circumferentially constrains the bending section 1012 of the instrument channel tube body 101, preventing the tension side 1012b of the instrument channel tube body 101 from bulging outwardly during bending and preventing excessive stress on the compression side 1012a from causing inward collapse, which would otherwise compromise the patency of the instrument channel.

According to a preferred embodiment, a pitch of the coil spring tube 102 is less than or equal to a diameter of the instrument channel tube body 101. Preferably, the coil spring tube 102 is a closely wound spring, i.e., a spring having a relatively fine pitch, as illustrated in FIG. 2. Specifically, the diameter of the instrument channel tube body 101 is 2 to 3 mm, and the pitch of the coil spring tube 102 is 0.1 to 1 mm. The pitch of the coil spring tube 102 refers to the axial distance, measured along the mean coil diameter, between corresponding points on two adjacent active coils, excluding the inactive (support) coils.

In the instrument channel tube of the present preferred embodiment, the pitch of the coil spring tube 102 is less than or equal to the diameter of the instrument channel tube body 101. When the instrument channel tube body 101 transitions between bent and straightened states, this prevents the compression side 1012a and the tension side 1012b of the bending section 1012 from becoming caught in the gaps between coils of the coil spring tube 102, which would otherwise cause the coil spring tube 102 to cut into or rupture the wall of the instrument channel tube body 101.

According to a preferred embodiment, a first strength of the coil spring tube 102 at the bending section 1012 is greater than a second strength of the coil spring tube 102 at the non-bending section 1013; and/or a first pitch of the coil spring tube 102 at the bending section 1012 is less than a second pitch of the coil spring tube 102 at the non-bending section 1013. Preferably, a first thickness of the coil spring tube 102 at the bending section 1012 is greater than a second thickness of the coil spring tube 102 at the non-bending section 1013, such that the first strength of the coil spring tube 102 at the bending section 1012 is greater than the second strength of the coil spring tube 102 at the non-bending section 1013.

In the instrument channel tube of the present preferred embodiment, because the bending section 1012 has the compression side 1012a and the tension side 1012b, stress concentration occurs in the wall of the instrument channel tube body 101. By providing the first strength of the coil spring tube 102 at the bending section 1012 that is greater than the second strength of the coil spring tube 102 at the non-bending section 1013, and/or the first pitch of the coil spring tube 102 at the bending section 1012 that is less than the second pitch of the coil spring tube 102 at the non-bending section 1013, the coil spring tube 102 at the bending section 1012 provides a greater constraining force to the instrument channel tube body 101, preventing deformation of the instrument channel tube body 101 and thereby reducing the risk of damage to the instrument channel tube body 101 due to stress concentration caused by bending.

According to a preferred embodiment, a second adhesive layer is further disposed between the coil spring tube 102 and the connector head 201, or between the coil spring tube 102 and the instrument channel tube body 101. Preferably, the second adhesive layer may be a bonding layer formed by adhesive. The second adhesive layer is not shown in the drawings. Specifically, when the instrument channel tube body 101 is sleeve-fitted over the exterior of the connector head 201, after the coil spring tube 102 is sleeve-fitted over the exterior of the instrument channel tube body 101, adhesive can be dispensed between the coil spring tube 102 and the instrument channel tube body 101 by means of adhesive dispensing, with the adhesive forming the second adhesive layer to fixedly connect the coil spring tube 102 and the instrument channel tube body 101. Similarly, when the connector head 201 is sleeve-fitted over the exterior of the instrument channel tube body 101, the coil spring tube 102 may also be fixedly connected to the connector head 201 via the second adhesive layer.

In the instrument channel tube of the present preferred embodiment, the coil spring tube 102 is fixedly connected to the connector head 201 or the instrument channel tube body 101, keeping the proximal end of the coil spring tube 102 in place at the sleeve-fit junction between the instrument channel tube body 101 and the connector head 201 and preventing the coil spring tube 102 from shifting during bending of the instrument channel tube body 101, which could otherwise cause the coil spring tube 102 to detach from the sleeve-fit junction between the instrument channel tube body 101 and the connector head 201. That is, the coil spring tube 102 of the present preferred embodiment still applies a preload to the sleeve-fit junction between the instrument channel tube body 101 and the connector head 201, thereby further ensuring the reliability of the connection at the junction between the instrument channel tube body 101 and the connector head 201.

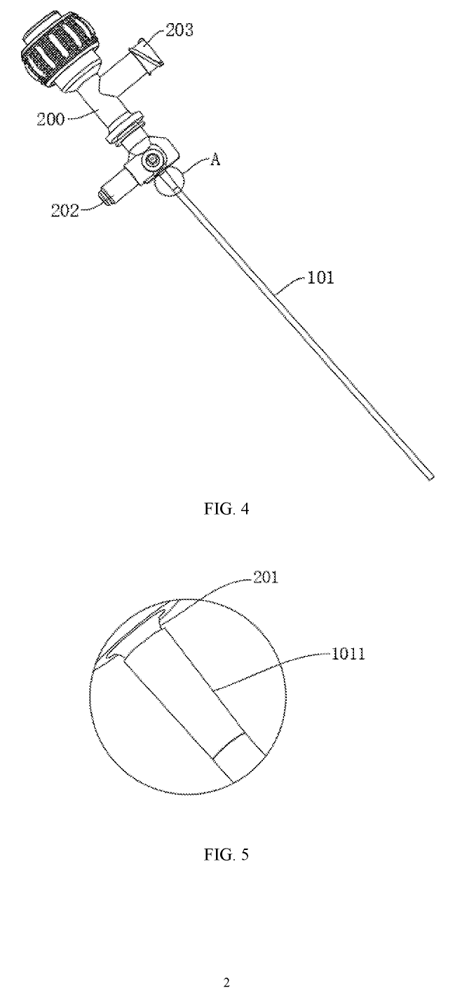

According to a preferred embodiment, the instrument channel tube further includes an overmold layer 103, as illustrated in FIG. 3. The overmold layer 103 is disposed over and encapsulates the exterior of the proximal end of the coil spring tube 102, as illustrated in FIG. 3. Preferably, the overmold layer 103 may be formed by an injection molding process. In the instrument channel tube of the present preferred embodiment, the overmold layer 103 is disposed over the exterior of the proximal end of the coil spring tube 102. The overmold layer 103 further ensures that the proximal end of the coil spring tube 102 always remains at the sleeve-fit junction between the instrument channel tube body 101 and the connector head 201, enabling the coil spring tube 102 to still apply a preload to the sleeve-fit junction between the instrument channel tube body 101 and the connector head 201, thereby further ensuring the reliability of the connection at the junction between the instrument channel tube body 101 and the connector head 201.

According to a preferred embodiment, the overmold layer 103 extends from the proximal end of the coil spring tube 102 toward the distal end of the coil spring tube 102, and the overmold layer 103 extends at least to the distal end portion of the coil spring tube 102, as illustrated in FIG. 3. In the instrument channel tube of the present preferred embodiment, the overmold layer 103 extends at least to the distal end portion of the coil spring tube 102. The overmold layer 103 imparts a smooth outer surface to the instrument channel tube, preventing friction between other structures within the insertion section (such as pull wires) and the coil spring tube 102, which would otherwise cause damage to the other structures within the insertion section.

The instrument channel tube of the present embodiment is assembled with the connector head 201 on the insertion section as follows:

Step 1: The instrument channel tube body 101 is sleeve-fitted over the exterior of the connector head 201, and the instrument channel tube body 101 is bonded to the connector head 201 by means of adhesive dispensing.

Step 2: The coil spring tube 102 is sleeve-fitted over the exterior of the instrument channel tube body 101 with the proximal end of the coil spring tube 102 positioned at the sleeve-fit junction between the instrument channel tube body 101 and the connector head 201, and then the coil spring tube 102 is bonded to the instrument channel tube body 101 by means of adhesive dispensing.

Step 3: The overmold layer 103 is formed over the exterior of the coil spring tube 102 by injection molding.

The instrument channel tube assembled in the manner described above provides the advantages of robust connection stability and good sealing between the instrument channel tube body 101 and the connector head 201.

In another aspect, the present embodiment further provides an insertion section for use in an endoscope.

The insertion section of the present embodiment includes an insertion section body and an instrument channel tube 100. The instrument channel tube 100 is the instrument channel tube according to any one of the technical solutions of the present embodiment. An accommodation space is defined within the insertion section body and extends along an axial direction of the insertion section body. The instrument channel tube 100 is disposed within the accommodation space. The insertion section body is not shown in the drawings. Specifically, the insertion section body may refer to structures of the insertion section other than the instrument channel tube 100. The structure of the insertion section body may be the same as that of an insertion section body of an endoscope in the prior art, and will not be described in further detail herein.

The insertion section of the present embodiment, by virtue of incorporating the instrument channel tube according to any one of the technical solutions of the present embodiment, provides the advantages of simple assembly operation, good sealing, insensitivity to the shapes and dimensions of the instrument channel tube body 101 and the connector head 201, high reliability of the connection at the junction between the instrument channel tube body 101 and the connector head 201, and independence of the connection reliability at the junction between the instrument channel tube body 101 and the connector head 201 from bending of the insertion section.

In a third aspect, the present embodiment further provides an endoscope.

The endoscope of the present embodiment includes an insertion section and a handle. The insertion section is the insertion section according to any one of the technical solutions of the present embodiment. A three-way connector 200 is disposed within the handle. The three-way connector 200 has a first port 2011, a second port 2021, and a third port 2031. The first port 2011 is in communication with the connector head 201, the second port 2021 is in communication with the suction port 202, and the third port 2031 is in communication with the instrument port 203, as illustrated in FIGS. 1 through 4 and FIGS. 6 through 8. In FIGS. 1 through 4, the housing structure of the handle is not shown; only the three-way connector 200 within the handle housing is illustrated. The remaining structure of the handle may be the same as in the prior art, and will not be described in further detail herein.

The endoscope of the present embodiment may be a nephroscope, a bronchoscope, an esophagoscope, a gastroscope, a colonoscope, an otoscope, a rhinoscope, an oral endoscope, a laryngoscope, a colposcope, a laparoscope, an arthroscope, or the like. The present embodiment does not impose any specific limitation on the type of endoscope.

The endoscope of the present embodiment, by virtue of incorporating the insertion section according to any one of the technical solutions of the present embodiment, provides the advantages of simple assembly operation, good sealing, insensitivity to the shapes and dimensions of the instrument channel tube body 101 and the connector head 201, high reliability of the connection at the junction between the instrument channel tube body 101 and the connector head 201, and independence of the connection reliability at the junction between the instrument channel tube body 101 and the connector head 201 from bending of the insertion section.

It should be noted that as used herein, the terms "including," "comprising," or any other variation thereof are intended to cover a non-exclusive inclusion, such that a process, method, article, or apparatus that includes a list of elements not only includes those elements but may also include other elements not expressly listed or elements inherent to such a process, method, article, or apparatus. Without further limitation, an element defined by the phrase "comprising a..." does not preclude the presence of additional identical elements in a process, method, article, or apparatus that includes the element. In addition, it should be pointed out that the scope of the methods and apparatuses in the embodiments of the present application is not limited to performing functions in the order shown or discussed, and may also include performing functions in a substantially simultaneous manner or in a reverse order based on the functions involved. For example, the described methods may be performed in an order different from that described, and various steps may also be added, omitted, or combined. Furthermore, features described with reference to certain examples may be combined in other examples.

The foregoing descriptions are merely specific embodiments of the present disclosure, but the scope of protection of the present disclosure is not limited thereto. Any variations or substitutions readily conceivable by those skilled in the art within the technical scope disclosed by the present disclosure shall fall within the scope of protection of the present disclosure.

What is claimed is:

1. An instrument channel tube for use in an endoscope, comprising:
an instrument channel tube body having an instrument channel formed therein, a proximal end of the instrument channel tube body being sleeve-fitted with a connector head at a distal end of a three-way connector of the endoscope such that the instrument channel is in communication with a suction port and an instrument port on the three-way connector, a first adhesive layer being disposed between the connector head and the instrument channel tube body; and
a coil spring tube disposed at a sleeve-fit junction between the instrument channel tube body and the connector head, the coil spring tube maintaining an interference fit with the sleeve-fit junction between the instrument channel tube body and the connector head and applying a preload to the sleeve-fit junction between the instrument channel tube body and the connector head.

2. The instrument channel tube according to claim 1, wherein the proximal end of the instrument channel tube body is sleeve-fitted over an exterior of the connector head, and the coil spring tube is sleeve-fitted over an exterior of the instrument channel tube body.

3. The instrument channel tube according to claim 1, wherein at least one of the connector head and the proximal end of the instrument channel tube body has a tapered configuration, a diameter of the tapered configuration gradually decreasing in a direction from the proximal end toward a distal end,
wherein an inner diameter of a proximal end of the coil spring tube is less than an outer diameter of the tapered configuration, such that when the coil spring tube is sleeve-fitted over the sleeve-fit junction between the instrument channel tube body and the connector head, the coil spring tube at the tapered configuration is configured to be radially expanded.

4. The instrument channel tube according to claim 1, wherein the coil spring tube extends from the proximal end of the instrument channel tube body toward a distal end of the instrument channel tube body, and the coil spring tube extends at least to a bending section of the instrument channel tube body.

5. The instrument channel tube according to claim 4, wherein a pitch of the coil spring tube is less than or equal to a diameter of the instrument channel tube body.

6. The instrument channel tube according to claim 5, wherein a first strength of the coil spring tube at the bending section is greater than a second strength of the coil spring tube at a non-bending section; and/or
a first pitch of the coil spring tube at the bending section is less than a second pitch of the coil spring tube at the non-bending section.

7. The instrument channel tube according to claim 1, wherein a second adhesive layer is disposed between the coil spring tube and the connector head, or between the coil spring tube and the instrument channel tube body.

8. The instrument channel tube according to claim 7, further comprising an overmold layer disposed over and encapsulating an exterior of a proximal end of the coil spring tube, and
the overmold layer extends from the proximal end of the coil spring tube toward a distal end of the coil spring tube, and extends at least to a distal end portion of the coil spring tube.

9. An insertion section for use in an endoscope, comprising:
an insertion section body defining an accommodation space extending along an axial direction of the insertion section body; and
the instrument channel tube according to claim 1 disposed within the accommodation space.

10. An endoscope, comprising:
the insertion section according to claim 9, and
a handle having a three-way connector disposed therein, the three-way connector having a first port, a second port, and a third port, the first port being in communication with the connector head, the second port being in communication with the suction port, and the third port being in communication with the instrument port.

11. The instrument channel tube according to claim 2, wherein at least one of the connector head and the proximal end of the instrument channel tube body has a tapered configuration, a diameter of the tapered configuration gradually decreasing in a direction from the proximal end toward a distal end,
wherein an inner diameter of a proximal end of the coil spring tube is less than an outer diameter of the tapered configuration, such that when the coil spring tube is sleeve-fitted over the sleeve-fit junction between the instrument channel tube body and the connector head, the coil spring tube at the tapered configuration is configured to be radially expanded.

12. The instrument channel tube according to claim 2, wherein the coil spring tube extends from the proximal end of the instrument channel tube body toward a distal end of the instrument channel tube body, and the coil spring tube extends at least to a bending section of the instrument channel tube body.

13. The instrument channel tube according to claim 12, wherein a pitch of the coil spring tube is less than or equal to a diameter of the instrument channel tube body.

14. The instrument channel tube according to claim 13, wherein a first strength of the coil spring tube at the bending section is greater than a second strength of the coil spring tube at a non-bending section; and/or
a first pitch of the coil spring tube at the bending section is less than a second pitch of the coil spring tube at the non-bending section.

15. The instrument channel tube according to claim 2, wherein a second adhesive layer is disposed between the coil spring tube and the connector head, or between the coil spring tube and the instrument channel tube body.

16. The instrument channel tube according to claim 15, further comprising an overmold layer disposed over and encapsulating an exterior of a proximal end of the coil spring tube, and
the overmold layer extends from the proximal end of the coil spring tube toward a distal end of the coil spring tube, and extends at least to a distal end portion of the coil spring tube.
